(19) United States
(12) Patent Application Publication
(10) Pub. No.: US 2016/0298104 A1
(43) Pub. Date: Oct. 13, 2016

(54) METHOD FOR ELECTRICAL TREATMENT OF FLUID MEDIUM CONTAINING BIOLOGICAL MATTER AND A SYSTEM FOR ITS IMPLEMENTATION

(71) Applicant: DECANT TREATMENT SYSTEMS, LLC, Cherry Hill, NJ (US)

(72) Inventors: Leonid Moldavsky, Nazareth-Illit (IL); Matityahu Fichman, Haifa (IL)

(21) Appl. No.: 14/680,126

(22) Filed: Apr. 7, 2015

(51) Int. Cl. C12N 13/00

(52) U.S. Cl. CPC C12N 13/00

(57) ABSTRACT

A method for electrical treatment of a fluid medium with distributed therein cells of biological material is disclosed. The method is suitable for treatment of plant biological matter such as raw, dried, or powdered flowers, roots, algae, stems, peels, seeds, fruits and the like; animal sludge, municipal waste solids and water, including increase of methane production in digester, fish or poultry solid wastes. The method is also suitable for the purpose of extraction of intracellular matter (oil, fats, sugars, minerals, etc.) from the cells, as well as for killing bacteria, pathogen, viruses, for reduction of concentration of solids, Phosphorus, Nitrogen, microbes, or for eliminating unpleasant odor. The treatment comprises exposure the fluid medium with the distributed therein biological matter to discrete consecutive pulses, while the treated material is placed in a processing unit provided with electrodes emanating the pulses. After treatment the biological material can be separated by one of the known separation technologies (gravitation, centrifugation, pressing, filtration, decanter, ion membrane exchange) or left as it is with improved environmental properties and used as a fertilizer, as a bedding, as food, etc.

### FIELD OF THE INVENTION

The present invention refers to treatment of fluid medium containing biological matter, preferably liquids containing plurality of biological cells distributed therein. Among the fluid medium, which can be treated in accordance with the present invention are for example various liquids containing bacteria, pathogens, viruses, algae and any other biological matter.

More particularly the present invention refers to method for electrical treatment of such liquids to cause lysis of the biological cells, their destroying and extraction of biological intracellular material from the biological cells.

Even more particularly the present invention refers to method for electrical treatment of liquids based on causing electro-kinetic flow within the liquid and near the cells, such that the flow causes destruction of membranes of the biological cells distributed in the liquid.

The method for electrical treatment and the system for its implementation in accordance with the present invention are suitable for example for treatment of liquids containing biological matter for the purpose of disinfection and/or for improving of various environmental parameters of waste waters.

Still further examples of liquid suitable for electrical treatment in accordance with the present invention are liquid waste waters originated from various industrial and agricultural installations, municipal sewage, waste waters originating from slaughter houses, from mining installations, fracturing waste waters, waste waters originating from food industry, from cosmetic industry, from pharmaceutical industry; liquids containing plant matter such as raw, dried, or powdered flowers, roots, algae, stems, peels, seeds, fruits and the like; animal sludge, municipal waste solids and water, including increase of methane production in digester, fish or poultry solid wastes. It should be taken into consideration, however, that the present invention is not limited to liquids containing solely biological cells; it is as well would be suitable for extraction of intracellular matter from liquids containing oils, fats, sugars, minerals, etc.

The method for electrical treatment according to the present invention comprises exposing electro-conductive liquid containing biological matter to discrete electrical pulses emanated from electrodes.

In accordance with some embodiments of the present invention the electrical treatment can be carried out either in batch mode or in the continuous mode.

After the treatment the biological material can undergo separation of solids from liquids by one of known separation technologies (gravitation, centrifugation, pressing, filtration, decanter, ion membrane exchange) or left as it is with improved environmental properties e.g. sprayed on fields as fertilizer, used as bedding, food, etc.

### BACKGROUND OF THE INVENTION

There are known in the art various methods for electrical treatment of liquids containing biological matter by means of applying electrical current thereto.

In WO 2008155315 there is described a device for cleaning and sterilizing fluids, in particular water. The device comprises elongated tubular container having an inlet and an outlet and a couple of flat electrodes installed within the container so as to be in the flow path of the fluid treated. According to the patent at least one electrode is coated with porous ceramic coating on the side facing the opposite electrode. The device comprises also an impulse generator unit electrically connected to the electrodes and capable for applying to the fluid of pulsed coronal discharges with the field strength of at least 10,000,000 V/m.

In US 2012000782 there is disclosed a uniform electrical field dielectric barrier discharge reactor for purifying of air, sterilizing of fluids or treatment of waste material. The reactor comprises an electrode unit, a dielectric catalyst container and an insulated housing. The electrode unit comprises electrode plates with discharge needles distributed on the insulated plane frame structure.

In CN 102060357 there is disclosed electrolysis reactor for treatment of high salinity waste waters. The reactor is designed as a cylindrical tube through which passes central water inlet pipe. The reactor is provided with radially installed flat electrodes.

In CN 201623198 there is described cylindrical reactor for use in microbial fuel cell. The reactor is provided with a couple of flat electrodes immersed in the electro genesis substrate within the reactor.

In JP 7299464 there is described multipurpose water treatment tank for sterilizing, cleaning and electrolyzing water. The tank is designed as a vessel of cylindrical configuration. The vessel is provided with a couple of concentric circular electrodes mounted to a cover such that they face each other.

In CN 102437360 there is disclosed multi electrode microbial fuel cell comprising a housing accommodating therein detachable circular partition plates of different diameter and detachable circular electrode plates of different diameters. Both the circular plates and the circular electrodes divide the housing into cathode chamber and anode chamber and they can be disassembled.

In CN101187038 there is described reactor for fluorination and electrolysis, which comprises electro pads, negative and positive electrode terminals, negative and positive electrode fitted rods and a generator.

In U.S. Pat. No. 6,141,905 there is described process and apparatus for utilizing animal excrement. According to this invention an aqueous mixture containing solid feed excrements from animals is subjected to treatment with an alternating electric current at a frequency of a predetermined magnitude and for a predetermined period of time, when the mixture passes through a tubular reactor.

In U.S. Pat. No. 6,344,349 there is disclosed process and system for electrical extraction of intracellular matter from biological waste materials, e.g. animal and human compost. The process comprises preparation of a mixture of biological matter with electro conductive fluid and then passing thereof through a processor unit while exposing the mixture to series of electrical pulses separated by pauses. In the process disclosed in U.S. Pat. No. 6,344,349 the electrical parameters of the applied pulses are controlled in such a manner, that the fluid medium is exposed to consequent series of electrical pulses which energy is sufficient for piercing of holes into or perforating the cell membranes of the cells. By virtue of this provision the cells membrane is destroyed and intracellular matter which releases from the cells is collected.

Unfortunately this mechanism requires electrical pulses with relatively large energies. This requirement, in its turn, is associated with excessive heating of the fluid medium, which might be not desirable or in some cases even not allowed at all. To prevent or at least to reduce the heating of the liquid the supplied electrical pulses are controlled in such a manner that intervals between consecutive series of pulses are kept long enough to let the fluid medium to cool down. It can be readily appreciated that in some cases this might render the process of treatment too long.

Furthermore, in order to generate electrical pulses with energy sufficient for perforating the cell membrane a relatively large power supply unit would be required, which might render this method and the system for its implementation too expensive, cumbersome and inefficient.

Thus it can be appreciated that despite of many attempts to solve the problem of electrical treatment of liquids containing organic matter there is still needed a new and improved method and system which would be suitable for fast, efficient and inexpensive treatment of various liquid wastes and in particular would be suitable for the extraction of intracellular matter from fluid biological waste materials.

### THE OBJECTS OF THE INVENTION

The main object of the present invention is to provide for a new and improved method and system suitable for inexpensive, efficient and fast extracting intracellular matter from fluids containing organic matter by exposing thereof to electrical pulses.

The further object of the present invention is to provide for a new and improved method and system for extracting intracellular matter from fluids containing organic matter by exposing thereof to electrical pulses by inducing electro-kinetic flow in the fluids resulting in lysis of wall of the cells.

Still further object of the present invention is to provide a new and improved method and system for extracting intracellular matter from fluids containing biological cells by exposing thereof to electrical pulses capable to induce electro-kinetic flow in the fluid resulting in acceleration of the cells and establishing shear stress in vicinity of the cell walls, while the shear stress stretches the cell walls.

Yet another object of the present invention is to provide a new and improved method and system for extracting intracellular matter from fluids containing biological cells by exposing thereof to bipolar, discrete electrical pulses separated by pauses, while duration of a single pulse being equal or shorter than duration of a pause between two consecutive pulses, and duration of the single pulse being equal or longer than the time of acceleration of the cells.

Still further object of the present invention is to provide a new and improved method and system for extracting intracellular matter from electrically conductive fluids containing organic matter by exposing thereof to discrete electrical pulses, resulting in establishing electrical field having electrical strength of 30 V/m to 30,000 V/m.

Yet another object of the invention is to provide a new and improved method and system for extracting intracellular matter from electrically conductive fluids containing organic matter while said fluids being defined by electrical resistance of 0.05 Ohm·m-5 Ohm·m and said fluids have weight concentration of organic matter of 0.1%-10%.

The electrical pulses employed in the method of the present invention are defined by a very short time period and by a low electrical strength. This specific combination allows inducing the electro-kinetic flow resulting in establishing shear stresses in vicinity of the cell's wall. Those cells which initially were in rest start moving. At the beginning of the motion, when the cells accelerate the flow velocity is relatively high A shear stress establishes which is applied to the cell's wall. As soon as the shear stress exceeds ultimate tensile stress of the cells walls it ruptures the cell's wall and eventually causes lysis of the cells. This process requires relatively low energy. Furthermore, the electro-kinetic flow influences on the cell membrane and this could create liposomes by virtue of vesicle biogenesis. The liposomes can be used for destroying viruses and for drug delivery due to blood circulation to various parts of the body, thus enhancing vesicle uptake by target cells.

The intracellular material could be separated from solids by any known separation means, e.g. pressing, gravitational separation, centrifugation, filtration or other means.

### BRIEF DESCRIPTION OF THE DRAWINGS

FIGS. 1-2 show schematically examples of cell walls which can be treated in accordance with the present invention.

FIG. 3 shows schematically electrical double layer around a single cell

FIG. 4 shows schematically formation of electro-kinetic flow.

FIG. 5 depicts schematically stresses applied to the cell membrane upon establishing of electro-kinetic flow.

FIGS. 6-7 show examples of electrical pulses employed in the method of the present invention.

FIG. 8 shows parameters of electrical pulses employed in accordance with the method of the present invention.

FIGS. 9-10 show schematically front view and side view of a cell and forces applied thereto due to the electro-kinetic flow.

FIG. 11 depicts schematically a system for implementation of the method of treatment in accordance with the present invention.

FIG. 12 presents an example of electronic scheme employed in the system shown in FIG. 11.

FIG. 13 is a graph showing reducing concentration of fecal coliform bacteria due to the treatment in accordance with the present invention.

FIGS. 14-18 show results of treatment of algae cells for destroying their membranes.

### DETAILED DESCRIPTION OF THE INVENTION

Before explaining the present invention in more details some definitions will be given for various terms which will be used in the future through disclosure of the present invention.

Under the term “lysis” here will be understood destruction, destroying or any other termination of integrity of biological cell which is caused by tearing off of the cell's outside wall and results in liberation of intracellular matter and its exit from the cell.

Under the term “cell wall” here will be understood an outside wall that surrounds and separates biological cells.

Under the term “intracellular matter” here is understood content of an organic cell delimited by its outer wall.

Under the term “electro-kinetic” flow here will be understood flow of a liquid, in which the cells are distributed, while this flow is induced upon exposure the liquid to an electrical field.

Under the term rms here is meant “mean root square”

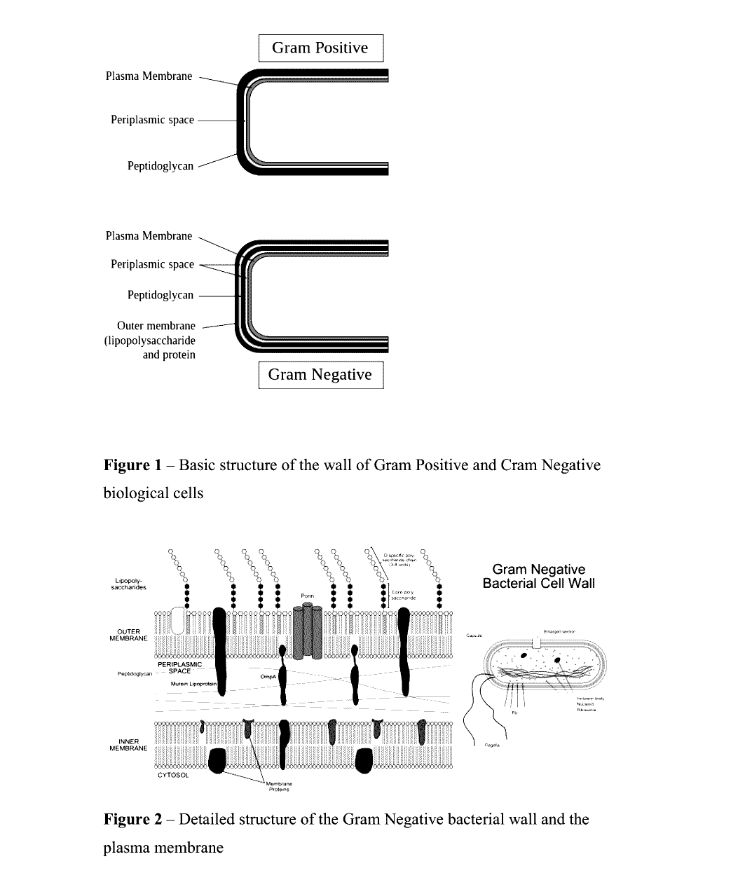

With reference to FIG. 1 and FIG. 2 it is schematically shown structure of bacteria cell, delimited by an outside wall.

In FIG. 1 there are shown two types of bacteria cell: Gram positive and Gram negative. The main difference between these two types is the structure of the outside wall. The structure of the Gram positive cell comprises a thick outer layer (Peptidoglycan) and inner plasma membrane. The Gram negative cell comprises two outer and inner plasma membranes. Between the membranes there is provided Periplasmic space (wall-less) or Peptidoglycan or other walls. Some examples of Gram negative bacteria comprise E-coli, Salmonella while to Gram positive belong for example streptococcus, staphylococcus.

With reference to FIG. 2 it is shown cell wall (or plasma membrane) consisting of both lipids and proteins. The fundamental structural feature of the membrane is the phospholipid bi-layer, which constitutes stable barrier separating aqueous compartments within the cell.

Proteins embedded within the phospholipid bi-layer carry out the specific functions of the plasma membrane, including selective transport of molecules and cell-cell recognition. The phospholipids are arranged in a bi-layer, with their polar, hydrophilic phosphate heads facing outwards, and their non-polar, hydrophobic fatty acid tails facing each other in the middle of the bi-layer. This hydrophobic layer acts as a barrier to all but the smallest molecules, effectively isolating the two sides of the membrane. Different kinds of membranes can contain phospholipids with different fatty acids, influencing on the strength and flexibility of the membrane, and animal cell membranes also contain cholesterol linking the fatty acids together and so stabilizing and strengthening the membrane.

It is known in the art to expose the liquid containing biological cells to electrical pulses for the purpose of destroying the biological cells. This method is known as electroporation.

The parameters of electrical field strength, E, used for bacteria electroporation are in the range of 100,000-2,500,000 V/m while the pulse duration is less than 0.001 s.

In the present invention is also employed exposure biological cells to electric pulses for the purpose of destroying the cells, nevertheless the electrical field strength of the pulses is several orders of magnitude lower in comparison with electroporation and it lies in the range of 30 V/m-30,000V/m. The range of the pulse duration is 0.00005-0.005 s.

It has been empirically revealed that it is possible efficiently destroy the cells by electrical pulses defined by much lower electrical field strength. This is possible by virtue of electro-kinetic flow induced in the liquid. The electro-kinetic flow causes establishing of shear stress in vicinity of the cell wall, which stretches it and eventually causes lysis of the cell as soon as the shear stress exceeds the ultimate tensile strength of the cell wall.

The bacteria cell membrane carries a net negative charge under most physiological conditions. This charge interacts with ions in the surrounding liquid, while generating different layers. Among them is the Stern layer which is a layer with ions charged oppositely to the surface charge of the cell wall. This layer is immobilized due to strong interaction with the cell wall. There is also the Gouy-Chapmen layer that consists of diffuse ions of net same sign as the Stern layer, but it is mobile.

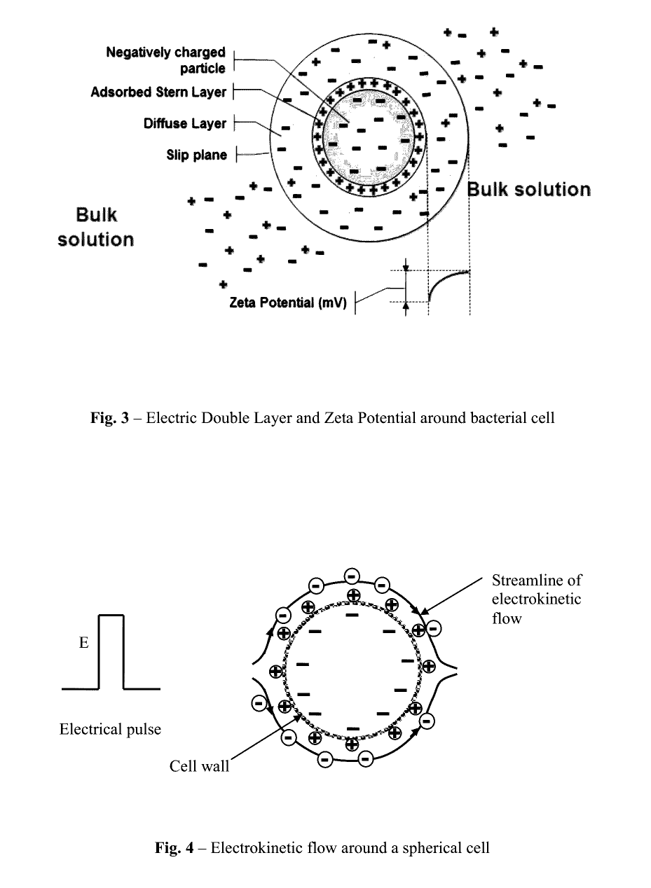

As shown in FIG. 3 together these two layers form the Electric Double Layer, EDL around the cell. When electrical field is applied to the liquid in which are distributed the cells to be destroyed their interaction with ions in the EDL induces electro-kinetic liquid flow as shown in FIG. 4. When alternative electrical pulses are applied to ionic liquid containing biological cells, the ions in the EDL start to move and their movement induces electro-kinetic flow in vicinity of the cell wall.

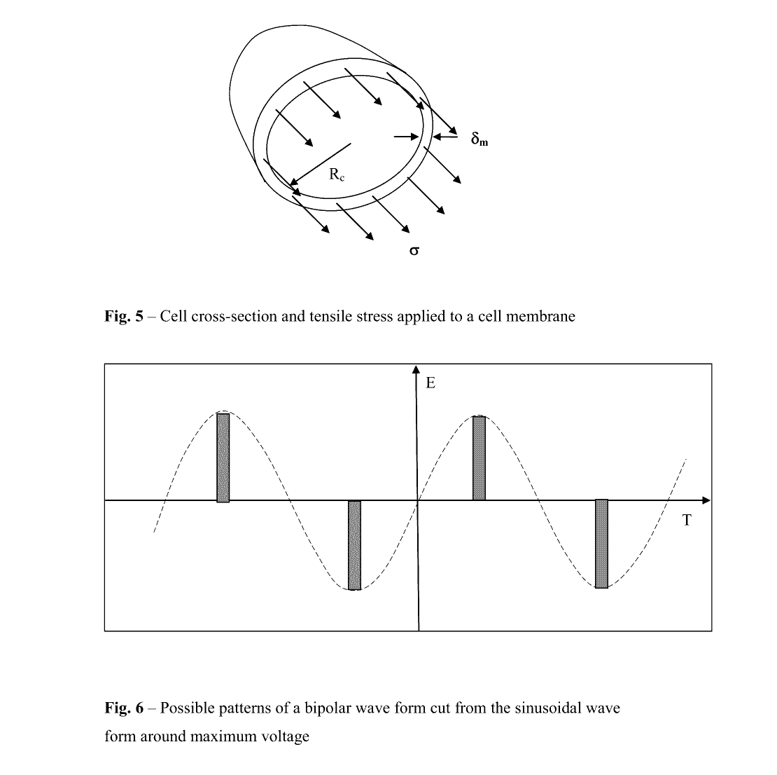

With reference to FIG. 5 it is schematically shown cross-section of a spherical bacteria cell, which has a radius R and an outer wall having a thickness δm. By virtue of the electro-kinetic flow a shear stress σ establishes in vicinity of the cell. This stress is applied to the outer wall as shown by arrows.

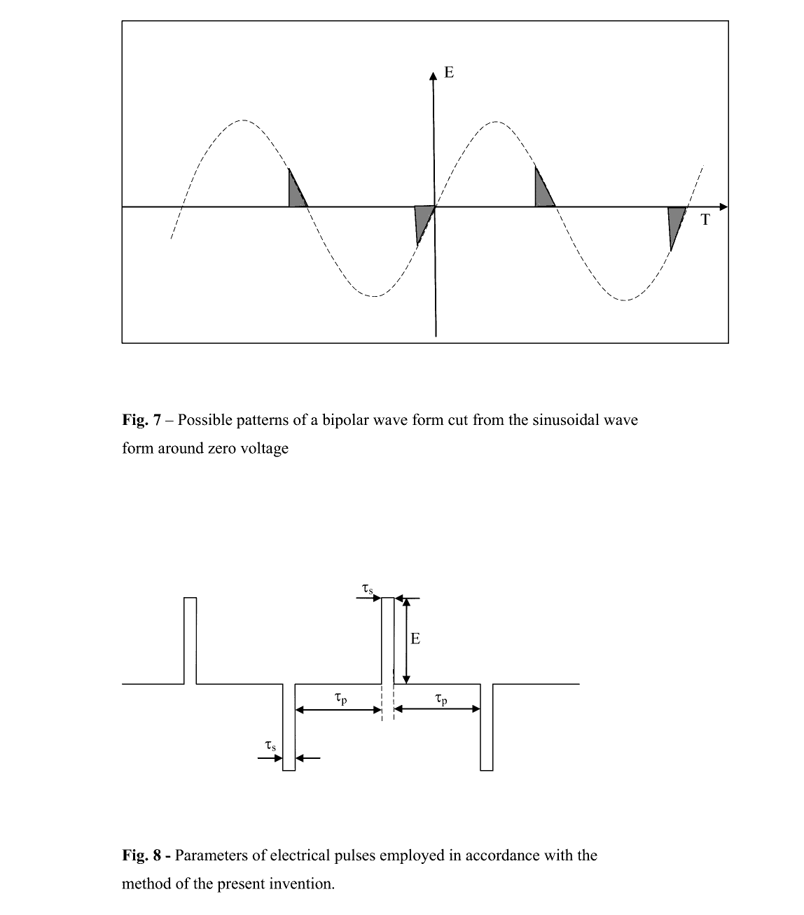

Referring now to FIG. 6 and FIG. 7 there is schematically shown possible patterns of a bipolar wave form and parameters of the cycled pulsed and paused electrical current, employed for inducing electro-kinetic flow in accordance with the present invention. The sinusoidal wave form is shown as a plot of amplitude (electrical field strength E) as function of time and the suitable pattern is shown as a hatched area on this plot. In practice the suitable pattern can be cut from the sinusoidal wave form as known in the art by modulation of electrical current supplied by a power supply unit.

Referring now to FIG. 8 it is shown schematically several consecutive pulses having rectangular shape, which were cut from bipolar sinusoidal alternating current. It should be understood, however that other shapes could be cut as well.

In FIG. 8 one can see two characteristic times i.e. duration of a single pulse (signal) which is denoted as τₛ, and duration of the pause between two consecutive pulses which is denoted as τₚ.

When electrical field is applied, first the electro-kinetic flow in a EDL is generated. At the beginning of the pulse, the cell due to its inertia is at rest. The electro-kinetic flow induces shear stress in vicinity of the outer cell wall due to viscosity of the liquid in which the cell is distributed. The shear stress causes acceleration of the cell during a characteristic time τ_{ac}.

During this time period the shear stress applied to the cell is maximal.

However as soon as the cell starts' moving with a steady state velocity, the shear stress reduces. The shear stress established during the acceleration period τ_{ac} stretches the outer wall and tears it off and brings to lysis of the cell as soon as the shear stress exceeds ultimate tensile strength of the outer wall. In accordance with the present invention in order to enable efficient lysis of the cell the pulse (signal) characteristic time, τₛ, should be equal or be less than the acceleration time, i.e.

τₛ≦τ_{ac}

This would be the first criterion, which should be satisfied in order to cause lysis of the cell by electro-kinetic flow in accordance with the present invention.

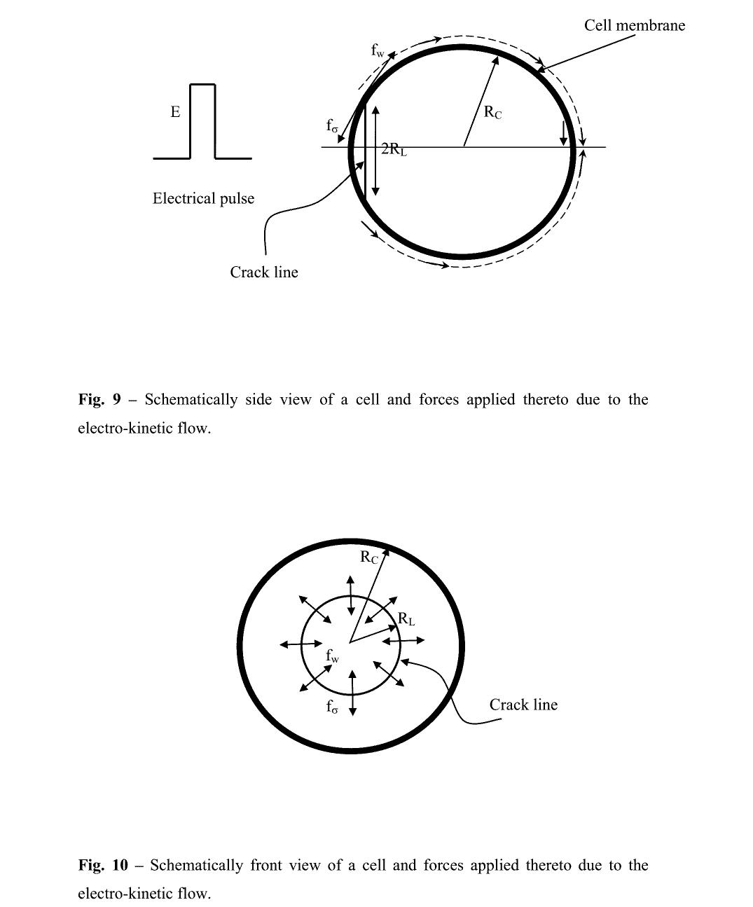

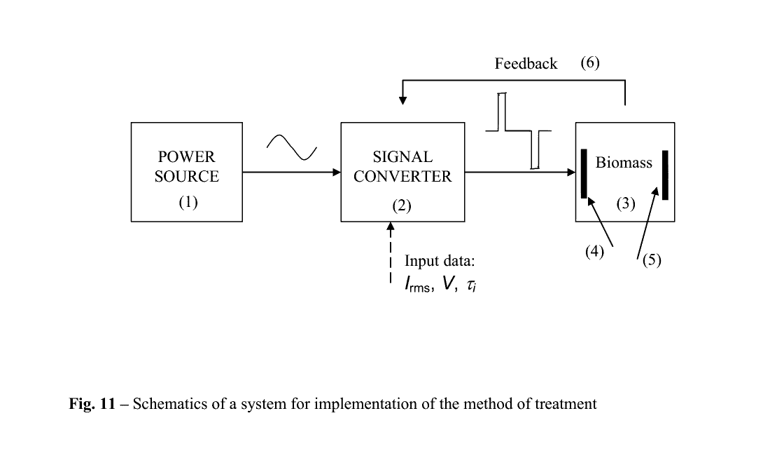

Now with reference to FIG. 5, FIG. 9 and FIG. 10 still further criteria will be explained, which should be also satisfied. The further criteria are derived with an assumption that the cell is spherical and that it is delimited by an annular outer wall.

In FIG. 9 and FIG. 10 is denoted a shear force Fσ acting on the cell and a tensile force Fw acting on the cell wall in response to the shear force. The tensile force is directed oppositely to the shear force. Furthermore it is denoted a radius of the cell Rc and a radius of rupture line RL.

Both forces remain equal until tensile force reaches ultimate tensile strength of the outer wall and further increase of the shear force results in rupture of the outer wall. Thus, the condition for rupture of the outer wall would be:

F_{w}=F_{σ}

From this expression it is possible to derive the radius, R_{L}, of the line of rupture of the cell as follows:

$${R}_{L} = \frac{S \cdot σ}{2 σ {δ}_{m} π}$$

where
S is area of the outer wall of the cell to which shear stress is applied,
σ_{w} is ultimate tensile strength of the area S
σ is shear stress applied to the area S

We can further assume that

S≈4πR_{c}²

And thus

$${R}_{L} \approx \frac{2 {R}_{c}^{2} \cdot {σ}_{w}}{ σ {δ}_{m}}$$

Thus the lowest value of R_{L} is equal to about diameter of lipid molecules, D_{lipid}, but it should be not more than radius of the cell, and therefore the second criterion for establishing the cell lysis is:

$$2 {R}_{c} > \frac{{R}_{c}^{2} \cdot {σ}_{w}}{σ {δ}_{m}} > 2 {D}_{lipid}$$

This criterion depends on different physical parameters of the biological system, like zeta-potential of a cell, temperature, ion's concentration, ultimate tensile strength of the cell wall and it also depends on the applied electrical field strength. The applied electrical strength and the ion concentration define the electrical current density. Therefore, the second criterion can be rewritten as a relationship between electrical field strength E and a density of electrical current J, or as electrical power E·J, which should be supplied to the cells during the treatment:

$$6 \cdot {10}^{3} \frac{VA}{{m}^{3}} < E \cdot J < 6 \cdot {10}^{6} \frac{VA}{{m}^{3}}$$

In the above expression the numbers are obtained empirically from the tests and m is meter, V—volt, A—amper.

In practice electrical pulses defined by electrical field strength of 30 V/m-30,000 V/m would be suitable for inducing electro-kinetic flow to cause rupture of walls of various biological cells and their lysis.

Furthermore it has been empirically revealed that after exposure the cells to a pulse the outer wall of the cells is stretched and a pause is required for relaxation of the cell wall before it is exposed to the next pulse. This relaxation or pause time, τₚ, should be equal or not shorter than the signal time and therefore the third criterion for achieving lysis of the cell in accordance with the present invention is:

τₚ≧τₛ

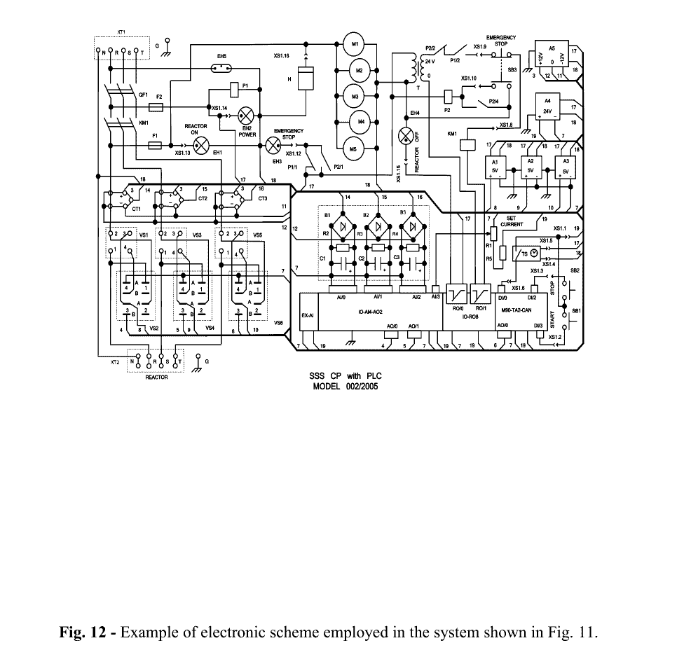

Now with reference to FIG. 11 and FIG. 12 a system for implementation of the method of the present invention would be disclosed. One should appreciate that for the sake of brevity there are depicted merely the most important units of the system, while in reality the system is provided with various necessary control and instrumentation equipment, mechanical and electronic equipment and mechanisms such as conduits, valves, connectors, ports, as would be necessary for transporting, mixing, and controlling flow between the units of the system.

A system shown in FIG. 11 comprises a power source unit 1, capable to generate and provide electrical power defined by bipolar sinusoidal pulses of alternating voltage V and alternating electrical current I. The power source unit is electrically connected with a signal convertor unit 2, which is capable to modulate and transfer the power so as to provide required pattern of the signal in accordance with electrical properties, e.g. resistivity of particular liquid to be treated. The convertor unit is electrically connected with a processor unit 3, which is provided with at least one couple of oppositely situated preferably flat electrodes 4, 5. The electrodes are disposed in the processor unit such that when the liquid with distributed therein biological cells (Biomass) is entered in the processor unit it is exposed to electrical pulses produced by the electrodes.

It is not shown specifically but should be appreciated that processor unit is provided with appropriate inlet and outlet port for entrance of liquid containing biological cells to be treated and for exit of the liquid with distributed therein intracellular matter liberated from the cells during the treatment. The power modulated by the convertor unit is supplied to the electrodes, which in response emanate electrical pulses into liquid to be treated. It is also shown schematically a feedback line 6, which enables adjustment of electrical parameters of the pulses.

As mentioned above the power source unit supplies sinusoidal electrical wave with specific voltage V and current I, depending on resistivity of the liquid media to be treated. In practice liquids having resistivity varied between 0.05 Ohm·m and 5 Ohm·m can be treated in accordance with the present invention. Therefore, the power should be modulated by the signal convertor unit 2 so as to supply a pulse with characteristic times, maximum amplitude voltage and amplitude current and with minimum rms current as might be required for treatment of particular biological material. By virtue of a feedback line 12 the minimum rms current is set by variation of the pulse characteristic time depending on the media electrical resistivity.

An example of electronic schematics, which is responsible for modulation of the power and supplying to electrodes pulses with required parameters, is depicted in FIG. 12.

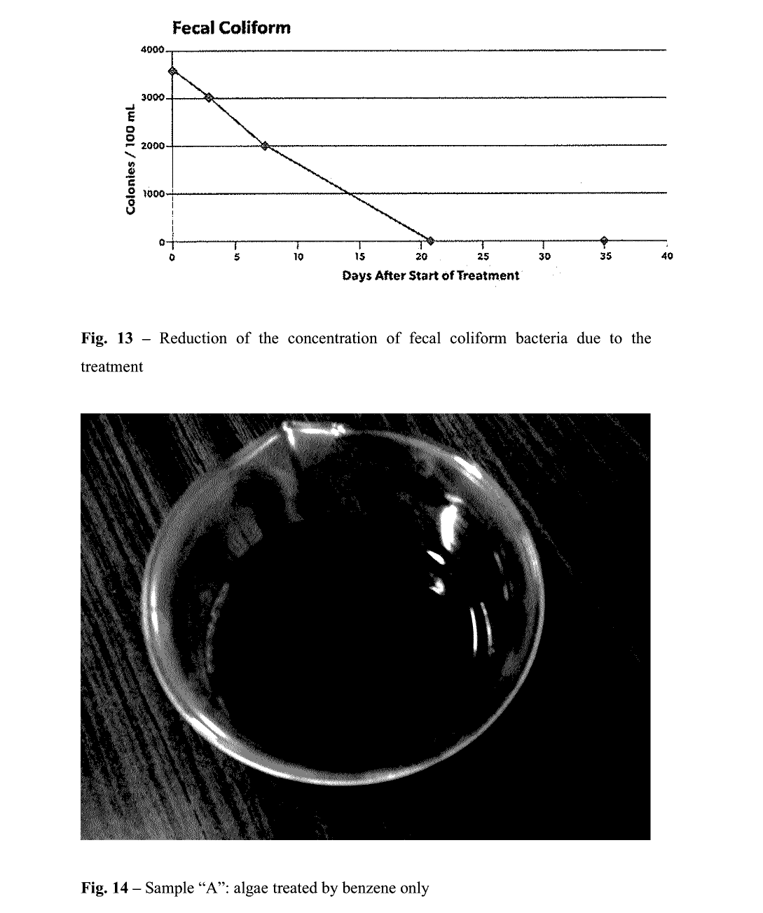

Now with reference to non-limited examples 1, 2 and 3 and to FIGS. 13-18 it will be explained how the present invention was implemented in practice.

### Example 1

### Treatment of Municipal Waste Water

Full scale operation unit was tested in close-loop system for treatment anaerobic digested sludge with concentration of solids about 1-2%. The sludge was exposed to electrical pulses cut from a standard sinusoidal wave having frequency of 60 kHz. The pulses were cut from the voltage 120 V supplied by the power source unit 1.

The maximum electrical strength of a single pulse was around E=250-400 V/m and the characteristic time, τₛ, was about 0.0006-0.001 s. The pause time, τₚ,

$${τ}_{p} = \frac{1}{2 \cdot 60 Hz} - {τ}_{s} ,$$

was about 0.0073-0.0077 s. The electrical resistivity of the media varied between 0.5 and 0.7 Ohm·m. Reduction of pathogens due to the electrical treatment is presented in FIG. 13. The electrical treatment lasted one week and then the treated sludge was left without treatment for additional two weeks. As one can see from the FIG. 13 the content of pathogens continued to diminish even after termination of the treatment.

### Example 2

### Sewage Treatment

The tests were carried out using an experimental lab unit that could supply pulses cut from standard sinusoidal wave of 120V having frequency 60 kHz as supplied by the power source unit. The pulse pattern was obtained by cutting thereof from the sinusoidal wave at close to zero voltage point. The maximum electrical strength of a single pulse was around E=250-400 V/m and the characteristic time were about 0.0006-0.001 s. In the second case the pulse pattern was obtained by cutting from the sinusoidal wave near the maximum voltage and the maximum electrical strength of a single pulse was around E=1,100 V/m and the pulse characteristic time, τₛ, was about 0.0002-0.0005 s. The pause time,

$${τ}_{p} = \frac{1}{2 \cdot 60 Hz} - {τ}_{s} ,$$

was about 0.0078-0.0081 s. The used pulse patterns are seen in FIGS. 6 and 7.

The treated material was the waste activation sludge (WAS) with solids concentration 1-3%. The material was treated during short periods, up to one minute and sent for analysis. In contrast to the first example it was batch treatment and not a close loop treatment.

In the first case, when the pulse pattern was as shown in FIG. 7 no significant reduction of e-coli and Fecal coliforms was observed. In the second case, when the pulse pattern was as shown in FIG. 6 the reduction of e-coli and Fecal coliforms was from 90 to 99%.

### Example 3

### Algae Treatment

The tests with algae distributed in water have been carried out with a purpose to destroy the cell membrane and to extract oils from the algae cells. The tests were performed using the both patterns of electrical pulses cut from standard sinusoidal wave of 220V having frequency 50 kHz as supplied by the power source unit, as in the example 2.

We have performed several tests that we can divide into three categories as follows:
“1”—treatment by benzene only;
“2”—heating to 90° C., benzene added after heating;
“3”—electrical treatment, at temperature 90° C., benzene added after treatment. The maximum electrical strength of a single pulse was around E=4,000-6,000 V/m and the characteristic time, τₛ, was about 0.0002-0.0003 s.

When we treated the algae solely with benzene we found no reaction. (Note: benzene was chosen as a product known for its extraction capabilities) In other words there was no oil liberation from the algae that naked eye could observe. The mixture looked the same before and after treatment. The picture labeled Sample “A” as seen in FIG. 14 is an example of our results; one can see that there is no change in algae appearance.

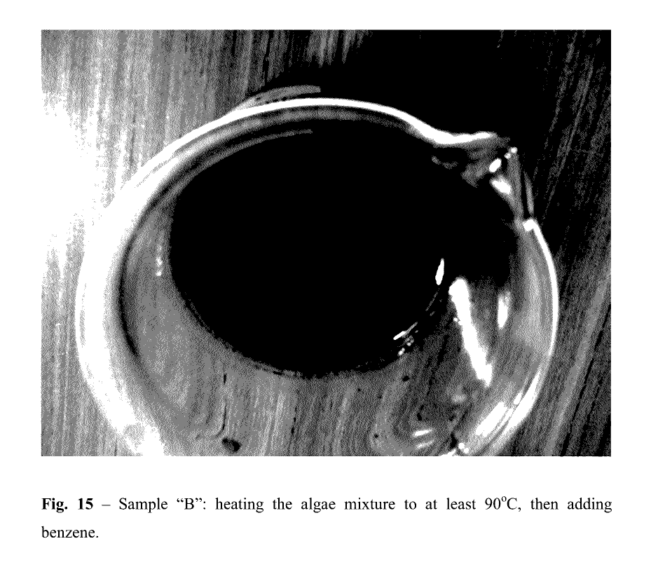

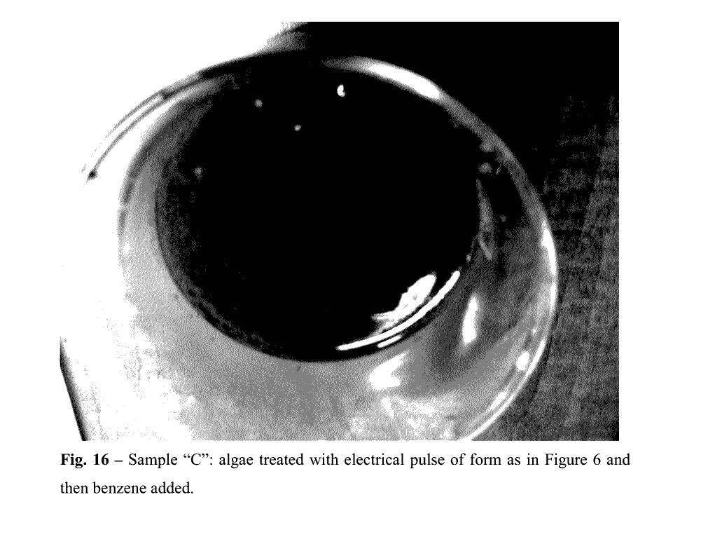

The next treatments involved first heating the algae mixture to at least 90° C., then adding benzene. At no point during this treatment did we observe any change in the appearance of the algae mixture. The results of this process are shown in FIG. 15, Sample “B”.

Finally, we conducted experiments in which we first exposed the algae mixture to electrical pulses, and then added benzene. The pulses of the pattern shown in FIG. 7 did not change the final results and appearance of algae mixture was as observed in sample A and B. However when the pulses with pattern shown in FIG. 6 have been used it was obvious to the naked eye that a significant difference occurred in appearance of the algae mixture approximately after few minutes of exposure. One can see spots of oil in the photograph and a clear separation of the oil from the algae mixture was apparent as can be seen in FIG. 16, Sample “C”.

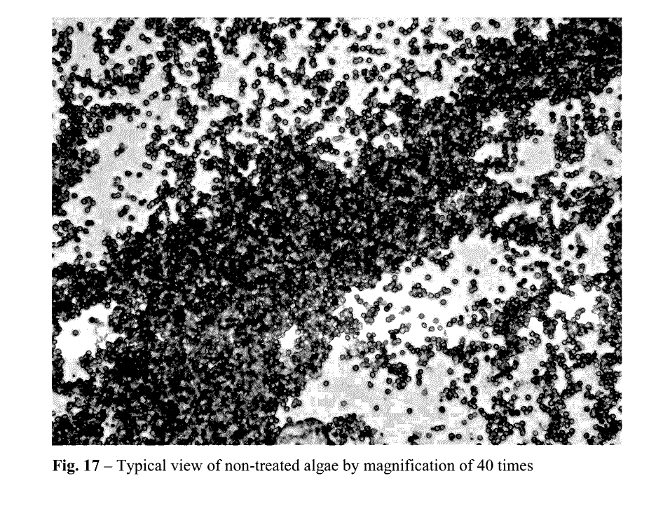

To enhance our study we used a microscope to better investigate appearance of the algae mixture as an indication of the impact of the treatment on the algae. In FIG. 17, is presented typical view of non-treated algae. The cells are mostly small, round and regular. This picture was taken at magnification of 40 times.

We then treated the algae by processing it in a centrifuge. What happened is that virtually all solid matter was separated and collected to one side and the water part was clear, meaning being free from all solids associated with the algae.

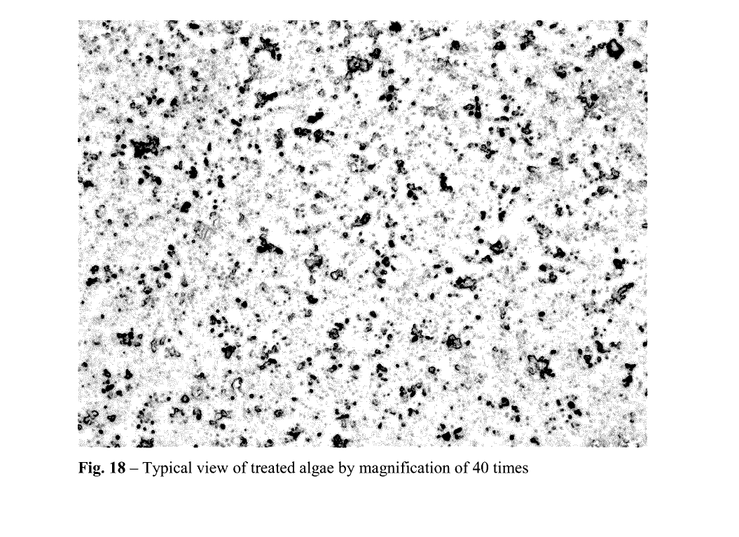

Next, we treated the mixture of algae with water by exposing it to electrical pulses in accordance with the present invention and again ran the mixture through the centrifuge. Remarkably, we could notice a significant difference in the “water” portion of the mixture that now obviously contained parts of the algae. Pictures of the liquid portion are shown below in FIG. 18.

The main conclusion from this experiment with the centrifuge and microscope is that the electrical treatment causes parts of the algae cells to break down and be left aside. No other extraction technique was used; neither did we estimate what percentage of algae was found in the liquid portion.

From the above examples it is evident that the method and system for electrical lysis of biological cells distributed in a liquid medium according to the present invention has advantages in terms of efficiency of the treatment and of convenience in exploitation.

It should be appreciated also that the present invention is not limited by the above described embodiments and that one ordinarily skilled in the art can make changes and modifications without deviation from the scope of the invention as will be defined below in the appended claims.

It should also be appreciated that features disclosed in the foregoing description, and/or in the foregoing drawings, and/or examples, and/or tables, and/or following claims both separately and in any combination thereof, be material for realizing the present invention in diverse forms thereof.

When used in the following claims the terms “comprise”, “contain”, “have” and their conjugates mean “including but not limited to”.

1. A method for electrical treatment of a fluid medium containing distributed therein biological cells comprising exposure the fluid medium to consecutive discrete pulses of electrical energy so as to induce an electro-kinetic flow of the fluid medium in vicinity of the biological cells, such that said electro-kinetic flow results in accelerating the biological cells, destroying their outer walls and liberation of a intracellular matter from the cells.

2. A method for electrical treatment as defined in claim 1, in which said electrical pulses are bipolar alternating pulses defined by a time of duration of a single pulse τₛ, as well as by a time of duration of a pause between two consecutive pulses τₚ and said fluid is exposed to said pulses during a period of time τ_{ac} during which the cells are accelerated by the electro-kinetic flow, wherein the above parameters satisfy the following conditions:
τₛ≦τ_{ac}, τₚ≧τₛ

3. A method for electrical treatment as defined in claim 2, in which said time of duration of the single pulse τₛ is 0.0001-0.005 s

4. A method for electrical treatment as defined in claim 2, in which said time of duration of the single pulse τₛ is 0.001-0.003 s.

5. A method for electrical treatment as defined in claim 1, in which said fluid medium comprises liquids defined by an electrical resistivity of 0.05 Ohm·m-5 Ohm·m

6. A method for electrical treatment as defined in claim 1, in which said electrical energy is defined by a field strength E, which is 30 V/m-30,000 V/m.

7. A method for electrical treatment as defined in claim 1, in which said electrical energy is defined by a field strength E, which is 30 V/m-3,000 V/m.

8. A method for electrical treatment as defined in claim 1, in which said electrical energy is defined by a field strength E, which is 500 V/m-30,000 V/m.

9. A method for electrical treatment as defined in claim 1, in which said electrical energy is defined by a rms electrical current density J, which is 200 Amp/m²-2000 Amp/m²

10. A method for electrical treatment as defined in claim 1, in which a weight concentration of the biological matter in the fluid medium is 0.1-10%.

11. A method for electrical treatment as defined in claim 1, in which said electro-kinetic flow causes establishing of a shear stress σ_{w} accelerating the cells and establishing of a tensile stress σ stretching the outer walls of the cells, while parameters of the pulses are selected in such a manner that a shear stress exceeds an elastic limit of the outer walls of the cells.

12. A method for electric treatment as defined in claim 1, in which said biological cells are selected from a group consisting of bacteria cells, pathogen cells and viruses cells.

13. A method for electric treatment as defined in claim 1, in which said cells are bacteria cells having lipid molecules in the outer wall, while said outer wall has a thickness δₘ and said lipid molecules have a diameter D_{lipid}

14. A method for electric treatment as defined in claim 11, in which destroying of the outer walls of the cells takes place along a circular line having a radius RL, and wherein the shear stress, the tensile stress, the thickness of the outer walls and diameter of the lipid molecules satisfy the following condition:
$$2 {R}_{c} > \frac{{R}_{c}^{2} \cdot {σ}_{w}}{σ {δ}_{m}} > 2 {D}_{lipid}$$

15. A method for electric treatment as defined in claim 1, in which said electrical pulses are defined by electrical field strength E and a density of electrical current J, satisfying the following condition:
$$6 \cdot {10}^{3} \frac{VA}{{m}^{3}} < E \cdot J < 6 \cdot {10}^{6} \frac{VA}{{m}^{3}}$$

16. A system for electrical treatment of a fluid medium containing distributed therein biological cells by exposing the fluid medium to consecutive discrete pulses of electrical energy, said system comprises a power source unit, which is electrically connected to a signal converter unit and to a processor unit, which is electrically connected to the signal converter unit, wherein the processor unit is provided with at least one pair of oppositely situated electrodes, capable to emanate pulses of electrical energy so as to induce an electro-kinetic flow of the fluid medium in vicinity of the biological cells, wherein said electro-kinetic flow results in accelerating the biological cells, destroying their outer walls and liberation of intracellular matter from the cells.

17. A system for electrical treatment as defined in claim 11, in which said power source unit is suitable for generation of discrete, consecutive electrical pulses of positive and negative polarities and said signal converter unit is capable to modulate the electrical pulses provided by the power source unit in such a manner that a time of duration of a single pulse τₛ, is shorter than a time of duration of a pause between two consecutive pulses τₚ and said signal converter unit is capable to control the power source unit in such a manner, that the induced electro-kinetic flow accelerates the cell during a period of time τ_{ac}, wherein the above parameters satisfy the following conditions:
τₛ≦τ_{ac}, τₚ≧τₛ

18. A system for electrical treatment as defined in claim 16, in which the processor unit is provided with at least one pair of electrodes made from a material selected from the group consisting of a metal, graphite and an electro-conductive plastic.

19. A method as defined in claim 1, in which the intracellular material is separated from the fluid medium by a separation technology selected from the group consisting of pressing, gravitational separation, centrifugation and filtration.
